(19) United States
(12) Patent Application Publication
(10) Pub. No.: US 2007/0043314 A1
(43) Pub. Date: Feb. 22, 2007

(54) ABDUCTION HINGE FOR A HIP STABILIZER

(75) Inventor: Max E. MOORE, Largo, FL (US)

Correspondence Address: ZARLEY LAW FIRM P.L.C., CAPITAL SQUARE, 400 LOCUST, SUITE 200, DES MOINES, IA, 50309-2350, US

(73) Assignee: RESTORATIVE CARE OF AMERICA INCORPORATED, St. Petersburg, FL (US)

(21) Appl. No.: 11/161,869

(22) Filed: Aug. 19, 2005

(51) Int. Cl. A61F 5/00

(52) U.S. Cl. 602/24

(57) ABSTRACT

An abductor hinge having a first and a second connector. The first connector having a hub with a projection that extends outwardly from the hub and a central bore that extends through the hub and the projection. The second connector has a hub juxtapositioned to the hub of the first connector, and having a recess that frictionally receives the projection. A central bore extends through the hub of the second connector to the recess and is in alignment with the bore of the first connector. A screw extends through the bores of the first and second connectors to secure the connectors together.

### BACKGROUND OF THE INVENTION

After hip surgery, it is desirable to control the degree of hip abduction. Likewise, for some congenital hip defects and hip dislocations, abduction is also necessary. A benefit of some hip abductors is the capability of adjustment to accommodate varying degrees of abduction. Hip stabilizers are well known in the art and some include adjustable hip abductors. However, such prior art devices are complicated devices having many parts. Further, these devices are limited in the degree of adjustment.

Therefore, an objective of the present invention is to provide an abduction hinge comprised of fewer parts that is more economical to manufacture.

A further objective of the present invention is to provide an abductor hinge that is capable of adjustment to an unlimited number of angles.

These and other objectives will become apparent from the following description of the invention.

### SUMMARY OF THE INVENTION

An abductor hinge having a first and a second connector. The first connector having a hub with a projection that extends outwardly from the hub and a central bore that extends through the hub and the projection. The second connector has a hub juxtapositioned to the hub of the first connector, and having a recess that frictionally receives the projection. A central bore extends through the hub of the second connector to the recess and is in alignment with the bore of the first connector. A screw extends through the bores of the first and second connectors to secure the connectors together.

### BRIEF DESCRIPTION OF THE DRAWINGS

FIG. 1 is a perspective new of a hip abductor hinge attached to a hip stabilizer;

FIG. 2 is a perspective view of a hip abductor hinge attached to a hip stabilizer and extended in an outward position;

FIG. 3 is a perspective view of an abductor hinge unassembled;

FIG. 3A is a perspective view of an abductor hinge completely assembled;

FIG. 4 is a side view of an abductor hinge while not engaged; and

FIG. 5 is a side view of an abductor hinge while engaged.

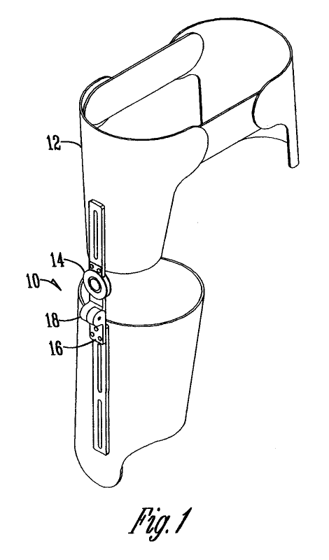

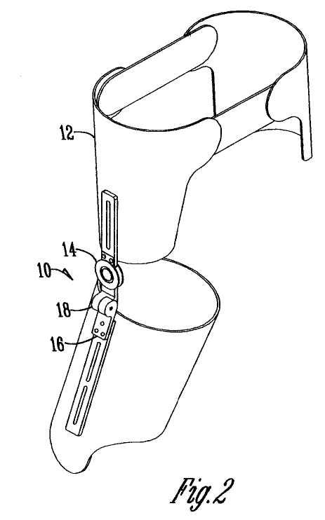

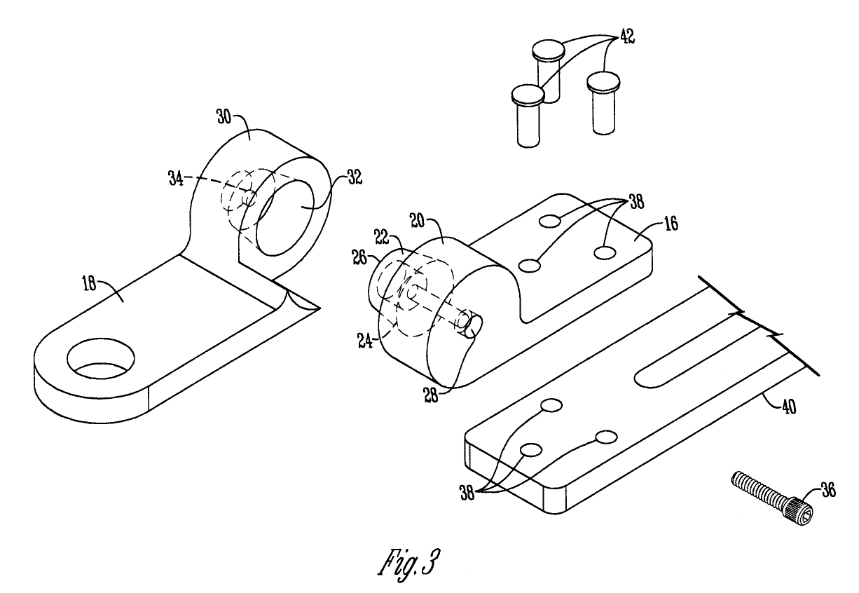

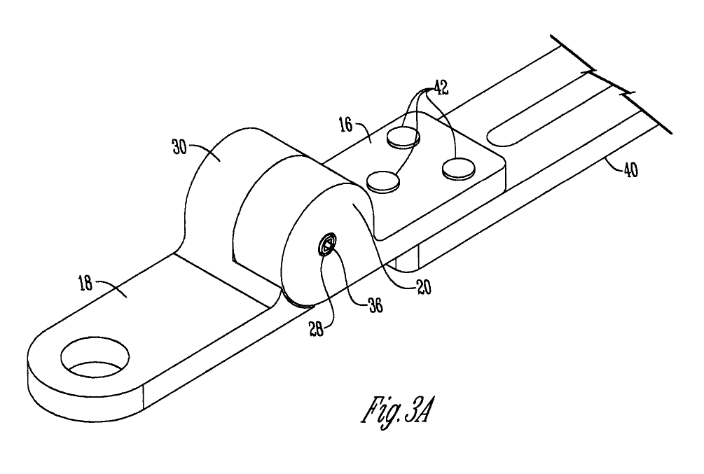

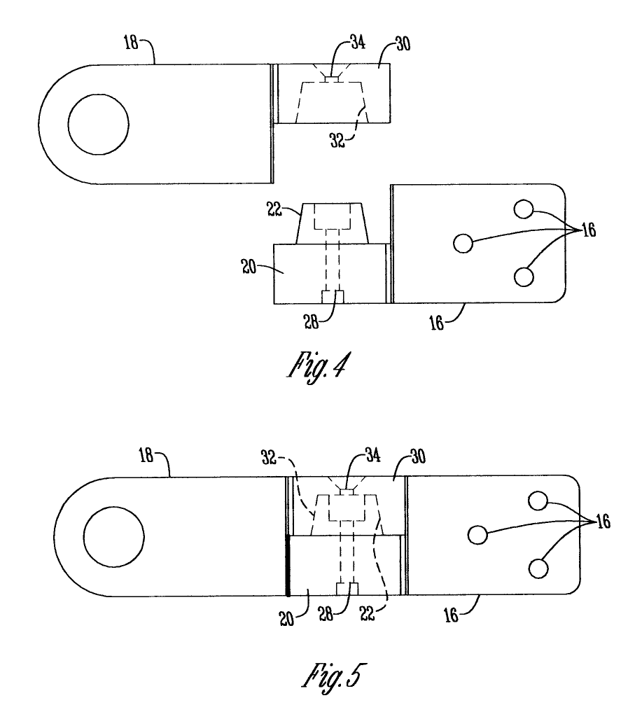

### DETAILED DESCRIPTION OF THE PREFERRED EMBODIMENT

Referring to the Figures, a hip abductor hinge 10 is attached to a hip stabilizer 12 and a flexion hinge 14. The abductor hinge includes a first connector 16 and a second connector 18. The first connector 16 has a hub 20 and a projection 22 that extends outwardly from the hub 20. Preferably, the projection 22 tapers as the projection 22 extends from its base 24 to its tip 26. The connector 16 has a hollow threaded bore 28 that extends from the hub 20 through the projection 22.

The second connector 18 has a hub 30 that is juxtapositioned with respect to the hub 20. Hub 30 has a recess 32 formed to receive projection 22. Extending through hub 30 is a hollow threaded bore 34 that is in alignment with bore 28. A screw 36 extends through bore 34 to bore 28 to connect first connector 16 to second connector 18.

Each connector has connecting bores 38 which are used to attach the hinge 10 to the hip stabilizer 12. An extending member 40 from the hip stabilizer 12 is connected to connectors 16 and 18 by an assembly screw or ribbet 42 that runs through aligned connecting bores.

In operation, projection 22 on hub 20 is fitted within recess 32 of hub 30 such that a co-efficient friction is created that overcomes torque motion when drawn together. The screw 36 is threaded through bores 34 and 28 to secure connector 16 to connector 18. To adjust the position of the first connector 16 in relation to the second connector 18, the screw 36 is rotated by a tool (not shown) such as an alien wrench such that projection 22 disengages from recess 32 so that connector 16 can be freely rotated. To relock the hinge 10, the screw 36 is rotated such that the projection 22 frictionally reengages recess 32 to overcome torque motion. Thus, a hinge has been shown that at the least meets all of the stated objectives.

The invention has been shown and described above in connection with the preferred embodiment, and it is understood that many modifications, substitutions and additions may be made which are within the intended broad scope of the invention.

What is claimed is:

1. An abductor hinge for a stabilizer, comprising:
a first connector having a first hub and a projection that extends outwardly from the first hub;
a second connector having a second hub with a recess that frictionally receives the projection; and
a screw that extends through the first and second hubs to secure the connectors together.

2. The hinge of claim 1 wherein the projection tapers inwardly from a base of the projection to a tip of the projection.
